(19) United States
(12) Patent Application Publication
(10) Pub. No.: US 2011/0213328 A1
(43) Pub. Date: Sep. 1, 2011

(54) Methods and Systems for Treatment of Neurological Diseases of the Central Nervous System

(75) Inventors: John G. Keimel, North Oaks, MN (US); William F. Kaemmerer, Edina, MN (US)

(73) Assignee: MEDTRONIC, INC., Minneapolis, MN (US)

(21) Appl. No.: 13/104,938

(22) Filed: May 10, 2011

Related U.S. Application Data
(62) Division of application No. 10/803,711, filed on Mar. 18, 2004.

(51) Int. Cl. A61M 25/00

(52) U.S. Cl. 604/500

(57) ABSTRACT

The present invention is directed to methods and systems for the treatment of inborn genetic errors or other defects that cause deficiencies of active enzymes or proteins within the cells of the central nervous system. Such methods and systems generally comprise an implantable catheter system designed for the chronic delivery of specially formulated proteins to intrathecal, intracerebroventricular, and/or intraparenchymal regions of the central nervous system. The invention has application in the neuropathic aspects of the broad category of lysosomal storage diseases. These genetic based diseases are the result of insufficient enzyme activity to catabolize specific substances, which thereby accumulate in the cellular lysosomes.

### TECHNICAL FIELD

The present invention relates generally to systems and methods for treating protein deficiency diseases, and more specifically to systems and methods of treating protein deficiency diseases using catheter devices to deliver enhanced protein replacement therapies to the central nervous system.

### BACKGROUND INFORMATION

Protein deficiency diseases are often the result of inherited errors or mutations of genes that are the basis for the creation of these proteins. Inborn errors of metabolism are a collection of these diseases, each caused by a mutation in a gene coding for a protein involved in the synthesis or catabolism of other proteins, carbohydrates, or fats. As a consequence of the gene mutation, the corresponding protein is absent or deficient in its level of activity. Subcategories of inborn errors of metabolism include amino acidopathies, urea cycle defects, lysosomal storage disorders, and fatty acid oxidation defects. Using lysosomal storage diseases as an example, the protein (enzyme) deficiency results in the toxic accumulation of substrates at the point of the blocked metabolic path, accumulation of toxic intermediates from an alternative pathway, or toxicity caused by a deficiency of products beyond the blocked point. The degree of metabolic deficiency, which is related to the degree of protein deficiency, is a major factor in the clinical manifestation (phenotype) and severity of the disease. Patients with complete absence or severe protein deficiency often die at a young age while patients with some limited protein activity may not show significant symptoms until adulthood. The degree of protein deficiency has been linked to specific mutations (alleles) of the responsible gene. For some diseases, there are numerous known alleles. Knowing a patient's specific allele thereby permits a projection of the disease course and also an opportunity for clinical intervention prior to degenerative consequences. Many of these protein deficiency diseases have an effect on the cells of the central nervous system. As a partial illustration of these diseases, a list of the lysosomal storage diseases for which there are substantial neurological involvement, along with the enzyme deficiency causing the disease, is shown in TABLE 1(a-f).

Because the enzymes needed to correct these diseases are known, one focus for treating inborn errors of metabolism has been to administer the missing enzyme to the patient suffering from the corresponding enzyme deficiency. Such “enzyme replacement therapy” (ERT) can be accomplished by administering an isolated or synthetic form of the enzyme (e.g., a recombinant protein) to the patient. Intravenous or other systemic administration of an enzyme as ERT can be effective in treating many disease symptoms in the internal organs and periphery. Enzymes, however, do not generally cross the blood-brain barrier, and these routes of administration have, therefore, not been effective at treating the neurological consequences of these diseases.

One way of addressing the problem of delivery of the deficient enzyme to the central nervous system (CNS) of patients suffering from these diseases is by gene therapy. Gene therapy involves genetically engineering the DNA coding sequence for the deficient enzyme into a non-viral or viral vector, then surgically injecting the vector into the brain, after which the cells transfected by the vector produce the missing enzyme and may secrete it to adjacent tissues. See Kmiec, “Gene Therapy,” American Scientist, 87(3): 240 (1999). To date, although this approach has been demonstrated to be feasible in numerous animal models of inborn errors of metabolism, it has not yet been proven effective in humans. Furthermore, recent cases involving gene therapy trials in humans (for the treatment of other disorders), including a three-year-old patient who developed leukemia during genetic therapy treatment for severe combined immunodeficiency (X-SCID), have resulted in a setback for such therapies. See Woo, “The Last Word: Researchers React to Gene Therapy's Pitfalls and Promises,” FDA Consumer Magazine, September-October (2000); Young, “‘Miracle’ gene therapy trial halted,” New Scientist, 14:30 (2002).

There have also been attempts to treat patients with enzyme deficient diseases by providing the needed enzymes through bone marrow transplants. See Hsu et al., “Niemann-Pick disease type C (a cellular cholesterol lipidosis) treated by bone marrow transplantation,” Bone Marrow Transplantation, 24:103-107 (1999); Yeager et al., “Bone marrow transplantation for infantile ceramidase deficiency (Farber disease),” Bone Marrow Transplantation, 26:357-363 (2000). Such attempts are based on the premise that undifferentiated stem cells originating from implanted bone marrow will develop into and replace the genetically-defective brain cells that cause a particular enzyme deficient disorder. The benefits of this technique for neurological disorders have not yet been shown.

Another way of replacing the deficient enzyme in lysosomal storage diseases is a therapeutic approach which reduces the initial creation of the substances (metabolites) that would otherwise accumulate in the lysosomes. Zavesca® (miglustat), a substrate reduction therapy, has now been approved in the United States and European countries for Gaucher disease, and has been proposed as having application in other lysosomal storage diseases in the same metabolic pathway. Cystagon® (cysteamine) is also being investigated as a substrate reduction therapy for infantile neuronal ceroid lipofuscinosis. Zavesca and Cystagon are small molecules that are believed to pass the blood-brain barrier. This type of therapy, however, is only applicable for those patients with some residual enzyme activity, and it requires a fine balance with the synthesis and catabolizing processes. Long-term benefit of substrate reduction therapy has not yet been demonstrated in humans. Possible long-term side effects are unknown, and this therapy is presently not recommended for growing children.

Another way of addressing delivery of the deficient enzyme to the CNS is by direct “manual” injection into the cerebral spinal fluid (CSF) of the patient, either at the spinal level (intrathecally) or into the intracerebral ventricles. In 1979, a case report described an early attempt to treat infantile Tay-Sachs disease in two infants by direct CNS injection of enzyme isolated and purified from human placentas [von Specht et al, “Enzyme replacement in Tay-Sachs disease,” Neurology, June; 29(6):848-54 (1979)]. In the first case, an initial intracerebroventricular injection followed by repeated intrathecal injections (via lumbar punctures) resulted in signs of clinical improvement in a 14-month-old infant (which was late in the disease course of this phenotype) including increased limb movement, cessation of food regurgitation, ability to lift the head, and smiling and laughing to appropriate stimuli. However, because no further improvement occurred after 10 weeks, treatment was discontinued and the infant expired. In the second case, treatment was initiated at 7 weeks of age and EEG recordings showed a normal pattern until age 10 months. At age 11 months, deterioration was observed, and treatment was discontinued at age 12 months. Analyses of blood samples from these patients showed the enzyme rapidly appeared in the serum following the injections into the CSF; also, post-mortem examination of brain tissue failed to provide any indication that the enzyme entered brain cells. While this case report has been interpreted by some as showing that “intrathecal ERT won't work,” others have concluded that this early attempt may have failed because the enzyme was not formulated in such a way so as to be readily taken up by cells [Dobrenis and Rattazzi, “Neuronal lysosomal enzyme replacement using fragment C of tetanus toxin,” Proc. Natl. Acad. Sci. USA, March 15; 89(6):2297-301 (1992)].

Lobel and Sleat, in U.S. Pat. No. 6,638,712, describe the use of a pump for administration of a therapeutic compound for the treatment of a specific lysosomal storage disease, late infantile neuronal ceroid lipofuscinosis. They suggest that such administration could include delivery of the therapeutic compound to intraventricular and intracranial sites. Based upon early human work, like that noted above for Infantile Tay-Sachs, the pumping of enzymes into the central nervous system does not overcome the inefficiencies of cellular uptake of the enzyme.

Several researchers have disclosed methods of modifying enzymes so as to enhance the uptake of enzymes by cells. Such modifications generally rely on a conjugation of the therapeutic enzyme with another species (generally a protein) or species fragment, having known transcytosis capability that serves as a transport aid across cell membranes.

Beliveau et al., in United States Patent Application Serial No. 20030129186, describe examples of enzyme compositions that enhance the transport through the blood-brain barrier. The intent is that this would permit intravenous injections that would ultimately reach the CNS. Even if effective in being transported across the blood-brain barrier, however, this method of systemic delivery would require the administration of large amounts of expensive enzymes with only a small percentage of these enzymes ultimately reaching the CNS.

The effectiveness of intrathecal delivery of enzyme replacement therapy for neuropathic lysosomal storage disease has been reported in a canine model of mucopolysaccharidosis [Kakkis, “Normalization of Carbohydrate Storage in Brain Tissue Using an MPS I Model,” Ninth International Congress on Inborn Errors of Metabolism, Sep. 3, 2003, Brisbane, Australia]. This experiment, conducted by BioMarin Pharmaceuticals, required repeated weekly intrathecal injections, which would present a major impediment for this to be used as ongoing therapy in humans.

Additionally, a potential problem in the treatment of these diseases is the possibility of toxic build-up and serious side effects of downstream metabolic byproducts upon initial treatment with the missing enzyme. This occurs when the sudden availability of the missing enzyme, and the presence of the accumulated substrate for it, results in the rapid production of downstream metabolic by-products of the previously blocked step, overwhelming the ability of the enzymes in the downstream pathways to perform their downstream steps. As a consequence, other metabolic intermediates can temporarily accumulate to levels sufficient to cause neurological damage.

Thus, methods for physically delivering such enhanced ERT to the central nervous system for long-term therapies remain an elusive challenge. Consequently, better methods for delivering enzymes, modified for enhanced cellular uptake, for ERT for neurological diseases would be of great benefit.

TABLE 1(a-f)
Lysosomal storage diseases with neurological involvement, and the
enzyme deficiency causing each disease

| | | | |
|---|---|---|---|
| Disease | Alternative Name | Enzyme | Neurological involvement |

a. Gangliosidosis (Sphingolipidosis)

| | | | |
|---|---|---|---|
| Gaucher's disease Types | Gaucher's disease | beta-glucosidase | Type II: dysphagia, palsy |
| II/III | | (glucocerebrosidase) | Type III: ataxia, seizures, |
| | | | dementia |
| Sphingomyelin lipidosis | Niemann-Pick disease | acid sphingomyelinase | Hypotonia, spasticity, rigidity, |
| | Type A | | mental retardation |
| Globoid cell | Krabbe's disease | galactocerebrosidase | cerebral atrophy, seizures |
| leukodystrophy | | | |
| Metachromatic | Metachromatic | arylsulfatase A | Rigidity, mental deterioration, |
| leukodystrophy | leukodystrophy | | convulsions; psychiatric |
| | | | symptoms in adult onset |
| | | | disease |
| Metachromatic | Metachromatic | saposin B | White matter lesions, |
| leukodystrophy without | leukodystrophy, variant | | cerebellar atrophy |
| arylsulfatase deficiency | form | | |
| Fabry's disease | Fabry's disease | alpha-galactosidase A | Autonomic dysfunction, |
| | | | neuropathic pain |
| GM1-gangliosidosis | Landing's disease | beta-galactosidase | Severe cerebral degeneration |
| GM2-gangliosidosis | Tay-Sachs disease | beta-hexosaminidase A | Psychomotor degeneration, |
| Type I | | | psychiatric symptoms |
| GM2-gangliosidosis | Sandhoffs disease | beta-hexosaminidase A | Cerebellar ataxia, dysarthria |
| Type II | | and B | |

b. Glycoprotein disorders

| | | | |
|---|---|---|---|
| Fucosidosis | Fucosidosis | alpha-L-fucosidase | Mental retardation, cerebral |
| | | | atrophy, seizures |
| alpha-Mannosidosis | Mannosidosis | alpha-D-mannosidase | Mental retardation |
| Types I/II | | | |
| beta-Mannosidosis | | beta-D-mannosidase | Hyperactivity, mental |
| | | | retardation |
| Aspartylglucosaminuria | Aspartylglucosaminuria | N-aspartyl-beta- | 3^{rd} most common genetic |
| | | glucosaminidase | cause of mental retardation |

c. Glycogen storage diseases

| | | | |
|---|---|---|---|
| Glycogen storage | Pompe's disease | alpha-glucosidase | Hypotonia |
| disease Type II | | | |
| Glycogen storage | Danon disease | LAMP-2 | Mental retardation, to variable |
| disease Type IIb | | | degrees |
| Glycogen storage | Andersen's disease | glycogen branching | Variable |
| disease Type IV | | enzyme | |

d. Mucolipidosis

| | | | |
|---|---|---|---|
| Mucolipidosis Type I | Sialidosis Type II | neuraminidase | Hypotonia, ataxia, seizures |
| Mucolipidosis Type II/III | I-cell disease | phosphotransferase | Severe psychomotor |
| | | | retardation |

e. Mucopolysaccharidosis

| | | | |
|---|---|---|---|
| Mucopolysaccharidosis | Hurler's syndrome, | alpha-L-iduronidase | Mental retardation |
| Type I | Scheie's syndrome | | |
| Mucopolysaccharidosis | Hunter's syndrome | iduronate-2-sulfatase | Hydrocephalus, mental |
| Type II | | | retardation, seizures |
| Mucopolysaccharidosis | Sanfilippo's syndrome | heparan-N-sulfatase | |
| Type IIIA | | | |
| Mucopolysaccharidosis | Sanfilippo's syndrome | alpha-N- | Hyperactivity, mental |
| Type IIIB | | acetylglucosaminidase | retardation, seizures, sleep |
| Mucopolysaccharidosis | Sanfilippo's syndrome | acetylCoA:N- | disturbances |
| Type IIIC | | acetyltransferase | |
| Mucopolysaccharidosis | Sanfilippo's syndrome | N-acetylglucosamine 6- | |
| Type IIID | | sulfatase | |
| Mucopolysaccharidosis | Morquio syndrome | galactose 6-sulfatase | Cervical myelopathy |
| Type IVA | | | |
| Mucopolysaccharidosis | Morquio syndrome | beta-galactosidase | |
| Type IVB | | | |
| Mucopolysaccharidosis | Maroteaux-Lamy | N-acetylgalactosamine | Cervical myelopathy, |
| Type VI | syndrome | 4-sulfatase | hydrocephalus |
| Mucopolysaccharidosis | Sly syndrome | beta-glucuronidase | Mental retardation, |
| Type VII | | | hydrocephalus, |
| | | | neurodegeneration |

f. Other Lysosomal Storage Disorders

| | | | |
|---|---|---|---|
| Cholesterol ester storage | Wolman disease | lysosomal acid lipase | lipid accumulation in glia |
| disease | | (acid cholesteryl ester | |
| | | hydrolase) | |
| Farber | Farber disease | acid ceramidase | mental retardation, seizures, |
| lipogranulomatosis | | | cerebral atrophy |
| Galactosialidosis | Schindler disease | N-acetyl-alpha-D- | mental retardation, seizures |
| Types I/II | | galactosaminidase | |
| Neuronal ceroid | Batten disease | palmitoyl protein | Most common |
| lipofuscinosis | | thioesterase | neurodegenerative disease in |
| | | | children; dementia, seizures |

### SUMMARY

The present invention is directed to methods and systems for the treatment of inborn genetic errors or other defects that cause deficiencies of active enzymes or proteins within the cells of the central nervous system. The invention has application in the neuropathic aspects of the broad category of metabolism diseases including lysosomal storage diseases. These genetically-based diseases are the result of insufficient enzyme activity to catabolize specific substances, which thereby accumulate in the neuronal lysosomes.

Bearing in mind the deficiencies in the present state of the art with regard to the treatment of genetically-based protein deficiencies of the central nervous system (CNS), it is an object of the present invention to provide improved methods of treating neurological diseases of the central nervous system, particularly lysosomal storage diseases, with enzyme replacement therapy (ERT). It is further an object to provide for systems by which to carry out such methods.

The present invention for protein delivery to the central nervous system also has application in the treatment of other neurological diseases, such as Fragile X Syndrome, which is a leading cause of genetic mental illness and which is now known to be the result of a specific protein deficiency. The present invention can provide for the delivery of this deficient protein and possibly benefit these patients. Other applications for this invention relate to the enhanced uptake of glial-derived neurotrophic factor (GDNF) by neurons that, in turn, can possibly provide for an improved treatment of Parkinson's disease.

The methods and systems of the present invention generally rely on one or more catheters to physically deliver therapeutic proteins across the blood-brain barrier (BBB) to the central nervous system for uptake by, for example, neuronal cells. Such protein delivery provides for treatment for a number of enzyme- and protein-deficient diseases. Acknowledging, however, that the blood-brain barrier is not the only obstacle to transcytosis of these proteins into cells of the central nervous system, as such transcytosis does not take place readily, the proteins must generally be “coaxed” into these cells by chemically modifying them with a transport aid.

Generally, the methods and systems of the present invention comprise an implantable catheter system to deliver therapeutic protein formulation intrathecally, intracerebroventricularly, and/or intraparenchymally to the central nervous system. In some embodiments, the methods and systems of the present invention further comprise a reservoir to store a quantity of a therapeutic protein formulation, as well as a pump to force the protein formulation through the catheter to a targeted delivery area. In some embodiments, one or both of the catheter and pump are implantable, i.e., surgically deposited inside the body of a patient. In some embodiments, the reservoir is integrated with the pump, as in, for example, the Medtronic SynchroMed pump. In some embodiments, the pump is programmable so as to be capable of altering the protein delivery rate in some predefined manner. This latter aspect permits a controlled dosing regimen.

In some embodiments, the present invention comprises an implantable drug pump+catheter system that permits a controlled and programmed release of specific proteins or enzymes that are deficient in the patient. The released enzymes or proteins in such embodiments can be conjugated or combined with carrier substances (transport aids) that thereby permit adequate transport and rapid uptake (e.g., endocytosis) of the active enzyme into central nervous system cells. In some embodiments, the protein substances are stored in a reservoir of the pump with an acidity level and formulation that reduces the degradation of the enzyme and proteins while stored in the reservoir. The catheter of such systems is designed to deliver the enzyme or proteins directly into the intrathecal or intracerebroventricular space, or directly into the parenchyma. Included in some such systems is a port that permits direct infusion of the enzyme or protein through the same catheter system. Additional or other embodiments may include a catheter system comprising an access port, which permits ease of access for repeated infusions of therapeutic proteins or enzymes.

Some embodiments of the present invention utilize intraparenchymal catheters (such as the Medtronic Model 8506 Intracerebroventricular Access Port and Catheter) to direct the delivery of a bolus of the enzymes or proteins that have been formulated for rapid uptake into the CNS cells. In some or other embodiments of the present invention, an implanted pump, similar to the Medtronic SynchroMed Infusion System (a peristaltic pump), or the Medtronic MiniMed 2007 System (a piston pump), is used for intrathecal, intracerebroventricular, and/or intraparenchymal delivery of therapeutic protein formulation.

The present invention, providing for the treatment of genetically-based protein deficiencies of the central nervous system, represents an advancement over the prior art in that it is presently safer than gene therapy approaches, it provides for enzyme replacement therapy (ERT) in cells of the central nervous system, it provides for the physical delivery of therapeutic proteins to the central nervous system, it provides for enhanced transcytosis of therapeutic proteins into cells, it provides for a programmable delivery of the therapeutic proteins, and it provides for the chronic delivery of therapeutic proteins for long-term therapies. The chronic delivery through an implanted catheter system, rather than repeated insertions into the CNS, has been shown to reduce the risk of infection. (Levy, R. Implanted Drug Delivery Systems for Control of Chronic Pain. Chapter 19 of Neurosurgical Management of Pain. New York, N.Y.: Springer-Verlag; 1997). Furthermore, the programmable delivery aspect of some embodiments of the present invention is beneficial in that it provides for treatment to be administered in varying dosages allowing for metabolic equilibration. This not only permits therapeutic levels but also permits cost effective amounts of proteins to be delivered, and without such considerations, dangerous levels of downstream enzymes or metabolites could ensue—jeopardizing the patient's therapy.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts an implantation of a pump and catheter system in a human body for the purpose of delivering therapeutic proteins for the treatment of protein deficiency diseases, according to one or more embodiments of the present invention;

FIG. 2 depicts a schematic representation of a human brain showing placement of the distal end of a catheter system in the intraparenchymal region of the central nervous system, according to one or more embodiments of the present invention;

FIG. 3 depicts a schematic representation of a catheter system suitable for delivering therapeutic proteins to intrathecal, intracerebroventricular, and/or intraparenchymal regions of the central nervous system for the treatment of protein deficiency diseases, according to one or more embodiments of the present invention;

FIG. 4 depicts a bifurcated catheter system, wherein the catheter system provides for the delivery of therapeutic proteins for the treatment of protein deficiency diseases to multiple locations or regions using a single pump system;

FIG. 5 depicts a top view of the implanted bifurcated catheter system, as employed in some embodiments of the present invention;

FIG. 6 depicts a generalized way in which a therapeutic protein is combined with a transport aid, with help from an optional linker species, to facilitate endocytosis according to some embodiments of the present invention;

FIG. 7 depicts a system, according to some embodiments of the present invention, wherein the system provides for the intrathecal, intracerebroventricular, and/or intraparenchymal delivery of therapeutic protein formulation for the treatment of protein deficiency;

FIG. 8 depicts intrathecal catheter placement according to some embodiments of the present invention;

FIG. 9 depicts a catheter system, according to some embodiments of the present invention, comprising an access port; and

FIG. 10 depicts the placement of the catheter system shown in FIG. 9, in accordance with some embodiments of the present invention.

### DETAILED DESCRIPTION

The present invention is directed to methods and systems for the treatment of inborn genetic errors or other defects that cause deficiencies of active enzymes or proteins within the cells of the central nervous system. The invention has application in the neuropathic aspects of the broad category of these protein deficiency diseases including lysosomal storage diseases. These genetic based diseases are the result of insufficient enzyme activity to catabolize specific substances, which thereby accumulate in the cellular lysosomes.

While most of the terms used herein will be recognizable to those of skill in the art, the following definitions are nevertheless put forth to aid in the understanding of the present invention. It should be understood, however, that when not explicitly defined, terms should be interpreted as adopting a meaning presently accepted by those of skill in the art.

“Neuropathic,” according to the present invention, means of or pertaining to neuropathy; of the nature of, or suffering from, nervous system disease.

A “disease,” as defined herein, is an impairment of health or a condition of abnormal functioning. This is closely related to a “disorder,” which is defined as a condition in which there is a disturbance of normal functioning.

“Proteins,” as defined herein, are macromolecular biological molecules made up of “amino acid” molecules (tryptophan, glycine, cysteine, etc.), wherein the isolated amino acid molecules each comprise an amino (—NH₂) group and a carboxylic acid (—C(O)ON) group. Linking amino acids together to form proteins or polypeptides requires a condensation reaction yielding peptide bonds. Complex proteins, comprising two or more polypeptide strands joined together by disulfide (—S—S—) and other bonds, also exist. “Enzymes” are macromolecules comprising any of numerous complex proteins that are produced by cells and generally act as catalysts in specific biochemical reactions (e.g., metabolic processes). In the description that follows, the more general term “protein” will be generally be used interchangeably with the term “enzyme”.

“Metabolism,” as defined herein, refers to the organic processes (in a cell or organism) that are necessary for life. As an example, the Krebs cycle is a series of enzymatic reactions in mitochondria involving oxidative metabolism of acetyl compounds to produce high-energy phosphate compounds that are the source of cellular energy. Metabolism may be either constructive or destructive (catabolism).

A “lysosome,” as defined herein, is an organelle found in the cytoplasm of most cells (especially in leukocytes and liver and kidney cells). They generally contain hydrolytic enzymes that can break down all polysaccharides, nucleic acids, and proteins as well as some lipids. They play a central role in cells' materials recycling and biosynthesis processes.

“Catabolize,” as defined herein, is something that is subject to catabolism, as in chemistry. Catabolism is the breakdown of molecules as a source of calories, and hence, a metabolic function that relies heavily on enzymatic processes.

“Implantable,” according to the present invention, generally refers to devices, e.g., catheters, that are inserted into a patient where they remain for a period of time that is generally in excess of two weeks.

A “catheter,” as defined herein, is a thin flexible tube inserted into the body to permit introduction or withdrawal of fluids or to keep the passageway open. According to the present invention, such a device is used to locally deliver therapeutic protein formulations to specific regions or organs within the body. A “catheter system,” according to the present invention, comprises a catheter and any additional devices that may be required to deliver therapeutic protein formulation (e.g., pumps, reservoirs, access ports, inlets, etc.).

“Parenchyma,” according to the present invention, is animal tissue that constitutes the essential part of an organ, as contrasted with, e.g., connective tissue and blood vessels. According to the present invention, parenchymal or intraparenchymal delivery or introduction, refers to the delivery or introduction of therapeutic protein formulation to the brain itself.

“Intracerebroventricular delivery,” in contrast to the delivery of therapeutic protein formulation to the parenchymal regions of the brain, refers to delivery of therapeutic protein formulation to the ventricular fluid-filled cavities within the brain, as opposed to the organ itself.

“Intrathecal,” according to the present invention, refers to the fluid-filled space between the thin layers of tissue that cover the brain and spinal cord. Drugs or other therapies can be injected into the fluid or a sample of the fluid can be removed for testing. Intrathecal delivery is delivery into or occurring in the space under the arachnoid membrane of the brain or spinal cord.

“Cerebrospinal fluid” (CSF), according to the present invention, is the fluid that fills the spaces in and around the brain and spinal cord, these spaces being the ventricles, spinal canal, and subarachnoid spaces. The principle source of CSF are the choroid plexi of the lateral, third and fourth ventricles and the volume generally varies between 10-20% of brain weight. The volume of CSF in humans is 140-150 ml, only 30-40 ml actually in the ventricular system, with a production rate of 21 ml/hr. The turnover rate of total CSF is species dependent and varies between approximately 1 hr for rat and 5 hr for human. The majority of the CSF is in the subarachnoid space, where the arachnoid membranes bridge the sulci of the brain, in the basal cisterns and around the spinal cord. CSF moves within the ventricles and subarachnoid spaces under the influence of hydrostatic pressure generated by its production. CSF cushions the brain, regulates brain extracellular fluid, allows for distribution of neuroactive substances, and is the “sink” that collects the waste products produced by the brain. Concentration of most molecules is greater in the brain than in the CSF, creating a physiological gradient between the two compartments. The continuous flow of CSF through the ventricular system and out over the surface of the brain provides a “sink” that reduces the steady-state concentration of a molecule penetrating into the brain and CSF. Few large molecules are typically able to gain entry into the brain cells via the CSF due to this bulk flow movement.

The “blood-brain barrier,” according to the present invention, is actually a mechanism that creates a barrier between brain tissue and circulating blood, serving to protect the central nervous system from pathogens within the blood circulatory system. Essentially, the endothelial cells that form the walls of the blood vessels within the brain are very selectively permeable.

“Protein deficiency diseases,” according to the present invention, are diseases that are caused by the absence or deficiency of one or more proteins. Enzyme deficiency diseases, as used herein, represent a subset of protein deficiency diseases wherein it is one or more enzymes that are absent or deficient in activity. As metabolism is highly enzyme-dependent, most inborn errors of metabolism are enzyme deficient diseases.

“Lysosomal storage diseases,” according to the present invention, are caused by a lack of enzymes that normally serve as catalyst for the breakdown of substances in the cells of the body. These enzymes are found in sac-like structures in cells called lysosomes. Lysosomes act as the “recycling center” of each cell, breaking down molecules into simple products for the cell to use to build new material. The lack of certain enzymes causes an accumulation within the cell of the substance that the enzyme would normally help eliminate. Abnormal storage causes inefficient functioning and damage of the body's cells, which can lead to serious health problems.

“Gene therapy,” as defined herein, generally refers to the therapeutic addition of genetic material to a patient via a viral or non-viral vector. Such genetic material, when introduced into a mammalian host, can express (i.e., code for) for proteins that were theretofore absent or deficient. Alternatively, the genetic material can be inserted into the patients DNA for more natural genetic expression. Finally, gene therapy can be used to suppress (i.e., turn off) the production of specific proteins. Such therapies rely heavily on knowing which genes are responsible for specific protein expression mechanisms.

“Substrate reduction therapy” (SRT), as defined herein, is a therapeutic approach which aims to reduce the synthesis of the substances in the cell and thereby provide equilibrium with a reduced enzyme activity available in lysosomal storage diseases.

“Enzyme replacement therapy” (ERT), according to the present invention, is generally a type of medical treatment for patients who lack an important enzyme; the missing enzyme is injected into the patient. Enzyme replacement therapies are, however, systemic treatments.

“Endocytosis,” as defined herein, is a process by which extracellular materials are taken up by a cell (e.g., cellular uptake). This contrasts to “exocytosis,” a process by which cellular material is discharged from a cell. While “transcytosis” generally describes the transport of materials through a cell membrane (encompassing both endo- and exocytosis), it is used synonymously with endocytosis herein.

“Lectins,” as defined herein, are any of several glycoproteins that act like specific antibodies but are not antibodies in that they are not evoked by an antigenic stimulus. “Endogenous lectins,” according to the present invention, are lectins that are derived internally by a patient's body.

“Streptavidin,” according to the present invention, is an tetrameric protein that is capable of binding to biotin (vitamin H), a cofactor required of enzymes that are involved in carboxylation reactions, via noncovalent interactions to form a “Streptavidin-biotin complex.”

“Conjugation,” according to the present invention, refers to the attachment of two or more species, wherein the attachment results from chemical or physical interactions. In some cases, a “linker species” is used to enable the conjugation. This linker species can be a molecule or molecular fragment, or it can be a functional group, e.g., a peptide linker linking two proteins or two amino acids via a peptide bond formed as a result of a condensation reaction between an amino functional group on one species and a carboxylic acid group on the other species.

An “Ommaya reservoir” is a device implanted under the scalp that is generally used to deliver drugs to the cerebrospinal fluid, the fluid surrounding the brain and spinal cord. A similar device called a “lumbar reservoir” is used to deliver drugs to the intrathecal space.

A “chronic implant,” according to the present invention, is one that is generally left in the body for a period of time that exceeds two weeks. “Chronic delivery,” according to the present invention, refers to the repeated delivery of a therapeutic agent or formulation over a period of time that is in excess of two weeks.

### Systems for Treating Neurological Diseases

In general terms, the present invention is directed to a system comprising a therapeutic protein formulation that has been modified for enhanced cellular uptake and whose delivery to central nervous system (CNS) cells is beneficial in treating neurological diseases of the central nervous system and comprises an implantable catheter system to physically deliver said protein formulation across the blood brain barrier. In some embodiments of the present invention, the catheter system comprises an inlet (e.g., injection) access port for introducing therapeutic protein formulation into the catheter system. In some embodiments, the catheter system further comprises an implantable reservoir to contain said protein formulation prior to delivery to said CNS cells and an implantable pump that pumps said protein formulation from the reservoir, through said at least one implantable catheter, and to at least one targeted region. In some embodiments, the reservoir is integrated with the pump. In some embodiments, the pump is programmable to allow for a variable delivery rate. In some embodiments, the integrated implantable pump+reservoir is refilled through an inlet access port.

In some embodiments of the present invention, intracerebroventricular catheters, such as the Medtronic Model 8506 Intracerebroventricular Access Port and Model 8770 Intracerebroventricular Catheter (Medtronic Inc., Minneapolis, Minn.), are used to direct the delivery of a bolus of the enzymes or proteins that have been formulated for rapid uptake into the CNS cells. In some or other embodiments, an intraparenchymal catheter, such as Medtronic Model 10541, is used for intraparenchymal delivery.

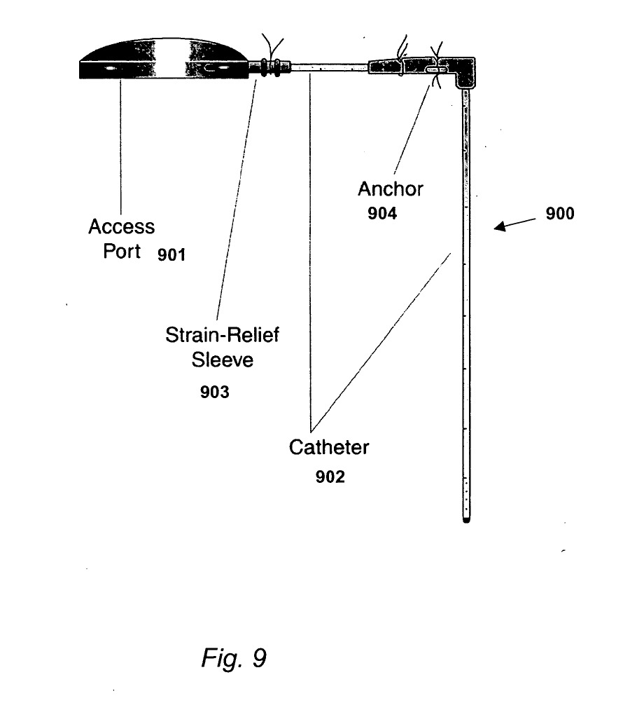

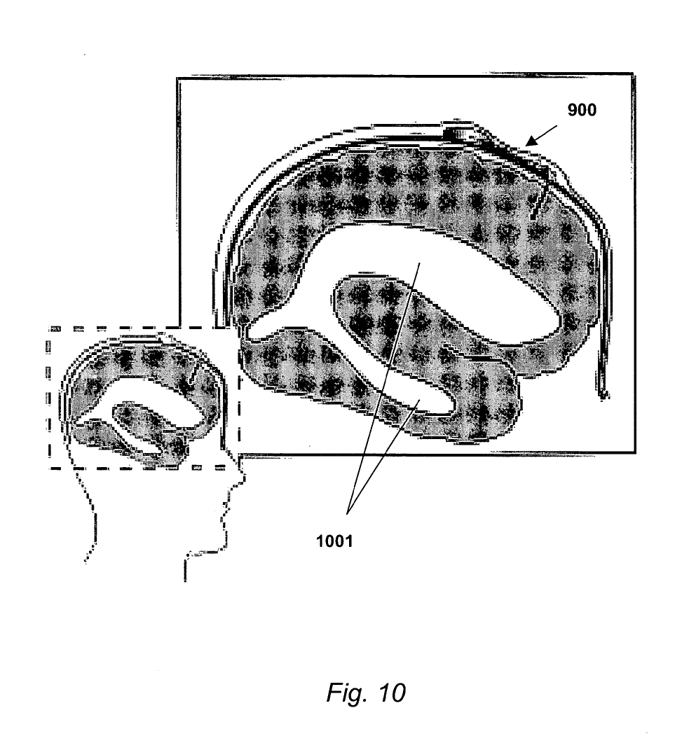

In some embodiments of the present invention, an implantable catheter comprising an access port is used. Such access ports can be of a wide variety of suitable inlet or injection ports. Shown in FIG. 9, is an example of one such suitable catheter system, wherein catheter system 900 comprises an access port 901 connected to a catheter 902 via a strain-relief sleeve 903 and further comprising an anchor 904 for anchoring the system to a patient as shown, for example, in FIG. 10. Referring to FIGS. 9 and 10, access port 901 is implanted on the top of the skull under the skin. Catheter 902 comes out of the port and runs parallel to the skull below the skin for a short distance, then goes into the head through a burr hole drilled in the skull, with the tip of the catheter penetrating into the brain tissue (for an intraparenchymal catheter). Alternatively, an intracerebroventricular catheter tip would penetrate through the brain tissue and into the cerebroventricals 1001, shown in a somewhat exaggerated manner. The catheter anchor is the subject of PCT Patent Application Publication Number WO2003090820.

In some embodiments of the present invention, an implantable pump, such as the Medtronic SynchroMed Infusion System or the MiniMed Model 2007 implantable pump, is used for intrathecal, intracerebroventricular, and/or intraparenchymal delivery of therapeutic protein formulation. Such systems comprise an implantable, programmable pump; an implantable catheter; and an external programmer. Suitable catheters include, but are not limited to, Medtronic InDura 1P Intrathecal Catheter Model 8709, and the InDura Free-flow Intrathecal Catheter Model 8711. Suitable pumps include, but are not limited to, the SynchroMed series of pumps by Medtronic Inc. Suitable models include, but are not limited to, 8626-18, 8626L-18, 8627-18, 8627L-18, 8626-10, 8626L-10, wherein all of these pumps have an integral reservoir, and wherein the pump is refilled by using a needle and syringe to inject the drug through the skin into the drug reservoir. Programming such pump+catheter systems to deliver a specific therapeutic protein formulation at a certain rate or programmed rate ramp can be done noninvasively with a Medtronic Model 8821 Programmer. Such programmable rates provide for a controlled dosing regimen, allowing for the avoidance of toxic side-effects of treatment.

Suitable catheter systems comprising pumps are described in commonly-assigned U.S. Pat. Nos. 6,093,180 and 6,594,880. The use of such systems for the general treatment of neurodegenerative disorders is described in commonly-assigned U.S. Pat. No. 5,814,014, and for the treatment of Alzheimer's disease in commonly-assigned U.S. Pat. Nos. 5,846,220; 6,056,725; and 6,503,242.

Alternatively or additionally, in some embodiments, a non-integrated reservoir may be used. Alternate catheter systems that may be used in accordance with the present invention for the delivery of enhanced therapeutic protein formulation to intrathecal, intracerebroventricular, and/or intraparenchymal regions of the central nervous system for the purpose of treating neurological diseases of the central nervous system include, but are not limited to, Ommaya reservoirs like those described in U.S. Pat. Nos. 5,222,982 and 5,385,582, and United States Patent Application Serial No. 20020142985.

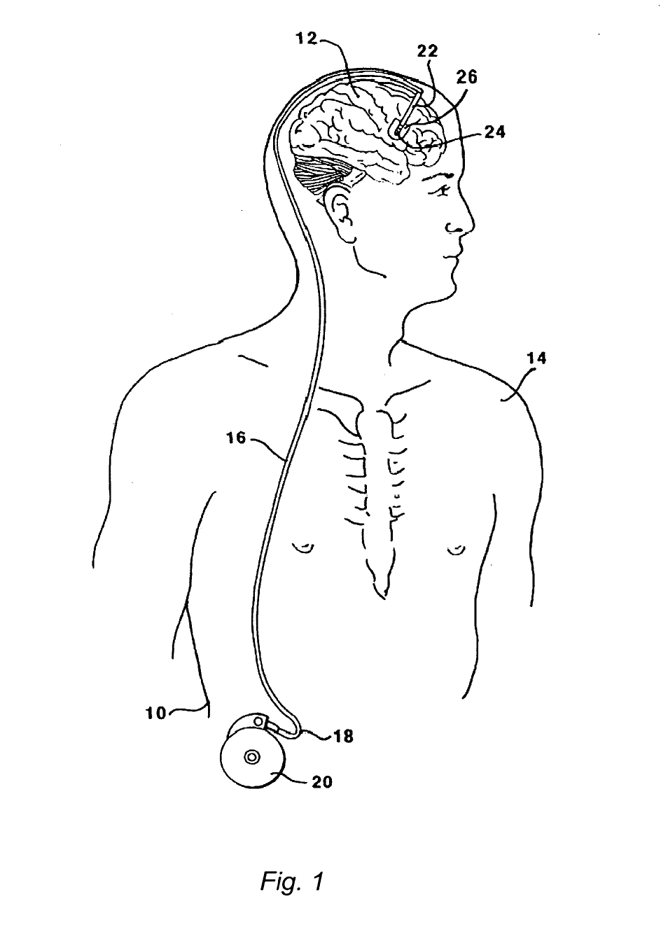

FIG. 1 depicts an embodiment of the present invention wherein catheter system 10 is used for the delivery of therapeutic protein formulation to an intracerebral (subset of intraparenchymal) region, and wherein the system 10 generally provides infusion of therapeutic protein formulation directly into the brain 12 in a human body 14. The catheter system 10 comprises a catheter 16 which has one end 18 coupled to an implanted infusion pump (IIP) 20 and a free distal end 22 for insertion into an organism, in this case, a human body 14. A catheter tip 24 is disposed at the extreme end of the distal end 22. The tip 24 has a rounded leading exterior surface to minimize tissue disruption during insertion.

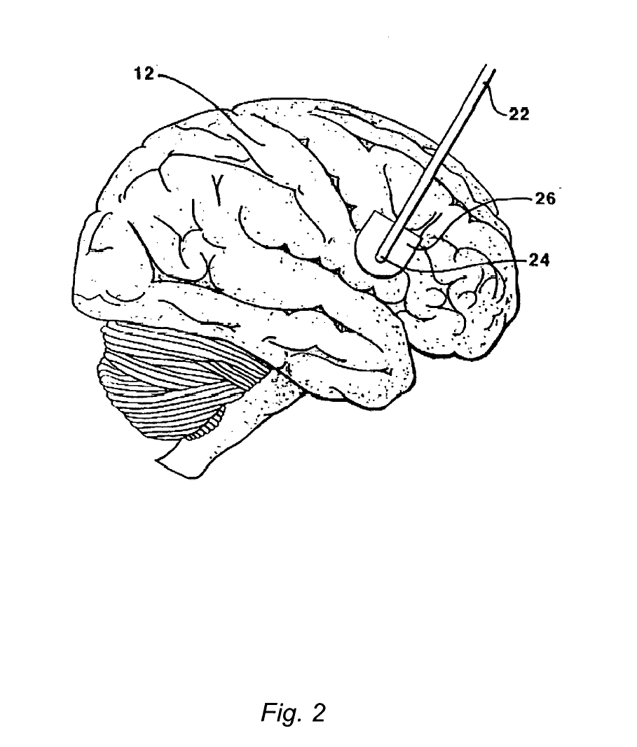

FIG. 2 depicts a schematic representation of a human brain showing placement of the tip of the catheter of the catheter system in the putamen, the outer part of the lenticular nucleus, according to at least one embodiment of the present invention. In the medical application portrayed in FIGS. 1 and 2, the distal end 22 is intracerebrally disposed so that the tip 24 projects into the putamen 26 of the brain 12. In the medical application depicted in FIGS. 1 and 2, the catheter tip 24 is positioned into the putamen 26 for retrograde access to the dopaminergic neurons contained within the retrorubral nucleus, substantia nigra, and ventral tegmentum. It should be understood by those skilled in the art that alternative locations could be used dependent on the specific disease being treated. As an example, for a disease such as Late Onset Tay Sachs, the catheter might be positioned directly into the cerebellum to treat that portion of the brain most affected by this lysosomal storage disease.

Still referring to FIGS. 1 and 2, the distal end 22 can be surgically implanted in the brain 12 using well known stereotactic placement techniques and the catheter 16 can be subsequently tunneled subcutaneously through the body 14 to the location in the body 14 where the IIP 20 will be implanted. The IIP 20 is ordinarily surgically implanted subcutaneously in the pectoral or abdominal region of the body 14. The IIP 20 may be any of a number of commercially available implantable infusion pumps such as, for example, the Medtronic SynchroMed pump, model 8611H, or other described herein.

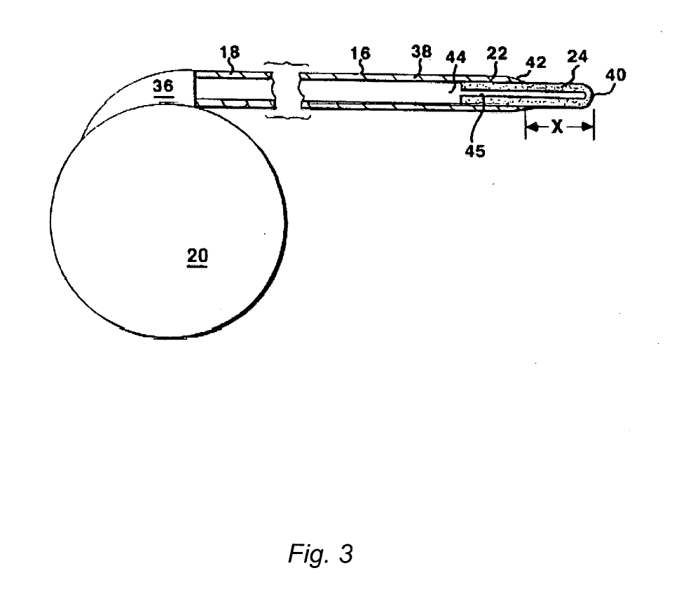

The detailed structure of the catheter system 10, as described above, may be further understood by reference to FIG. 3, which depicts a suitable catheter system in accordance with embodiments of the present invention. Catheter system 10 with the catheter 16 and the distal end 22 are shown in an enlarged half section in FIG. 3. The size of the catheter 16 and the distal end 22 are shown highly exaggerated for ease of illustration of the structure thereof and the full length of the catheter 16 is not shown for simplicity of illustration. The end 18 of the catheter 16 is coupled to the pump connector 36. The connection between the catheter 16 and the pump connector 36 is shown schematically in FIG. 3. It should be understood that the actual type of connection between the pump connector 36 and the catheter 16 will vary depending upon the particular type of IIP 20 utilized.

Referring to FIG. 3, catheter 16 comprises an elongated tubular portion 38 that extends from the pump coupling 36 and terminates in the distal end 22 and the tip 24. As noted above, the catheter tip 24 has a generally rounded leading exterior surface 40 to minimize tissue disruption during insertion. The tubular portion 38 has an externally tapered end surface 42 to again minimize tissue disruption during insertion. The catheter tip 24 has a generally tubular shape and is designed to fit snugly within the lumen 44 of the tubular portion 38. The catheter tip 24 has a lumen 45 to receive agent from the catheter lumen 44. The catheter lumen 44 and the external diameter of the catheter tip 24 are typically sized so that there is a zero tolerance therebetween. A snug fit is desirable to both maintain the position of the catheter tip 24 in relation to the tubular portion 38 and to discourage seepage of agent between the interface of the exterior of the catheter tip 24 and the interior surface of the tubular portion 38. Under certain conditions, however, the catheter 16 may be customized by moving the catheter tip 24 in relation to the tubular portion 38.

Still referring to FIG. 3, in some embodiments of the present invention, the catheter tip 24 is comprised of a porous material such as polysulfone hollow fiber like that manufactured by Amicon, although polyethylene, polyamides, polypropylene and expanded polytetrafluorethylene (ePTFE) are also suitable. The catheter tip 24 is typically porous along its entire length to enable agent to flow into the body 14. The typical pore size of this catheter is approximately less than or equal to about 0.22 microns (micrometers). Generally the maximum pore size is less than or equal to approximately 0.22 microns to prevent any derelict bacterial agents, that may be present inside the catheter 16, from entering into the body 14. Furthermore, at larger pore sizes, there is the potential for tissue in-growth that may restrict the flow of agents out of the catheter tip 24. By making the entire length of the catheter tip 24 porous, a more uniform volume distribution of agent is provided. It should also be clear to those skilled in the art that the pore size can be adjusted to allow for the delivery of a specific protein while still preventing ingrowth or preventing bacterial agents from entering into the body.

The catheter tip can use a single or multiple elution holes. Alternatively, the catheter tip 24 of FIGS. 1-3 dispenses agent in a nearly 360 degree pattern along the entire length of the catheter tip 24 that is exposed to the parenchymal target, represented in FIG. 3 by the length X. Herein, the length of the portion of catheter tip 24 that is exposed to the parenchymal target is represented by X. Length X may be custom selected by a physician at the time of insertion. To enable the physician to customize length X, the tubular portion 38 is typically comprised of a material that will expand in response to an external stimulus such as heat or a chemical solvent. When the tubular portion 38 expands in response to the external stimulus, the snug fit between the catheter tip 24 and the tubular portion 38 is relieved, and the physician may slide the catheter tip 24, with respect to the tubular portion 38, by hand to achieve the desired length X. The material from which the tubular portion 38 is comprised, is typically selected such that when the external stimulus is removed, the tubular portion 38 returns to its ordinary shape, thereby reestablishing the near zero tolerance fit between the tubular portion 38 and the catheter tip 24.

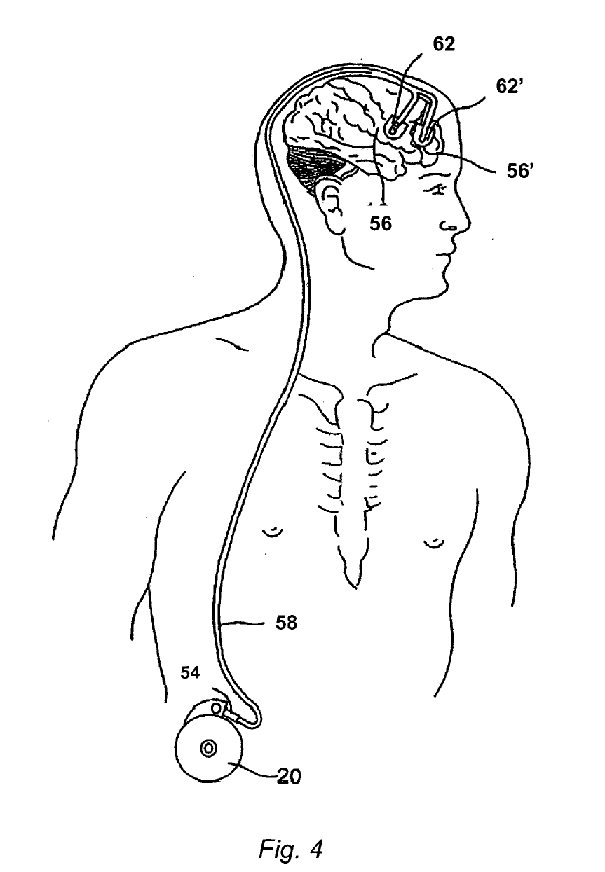

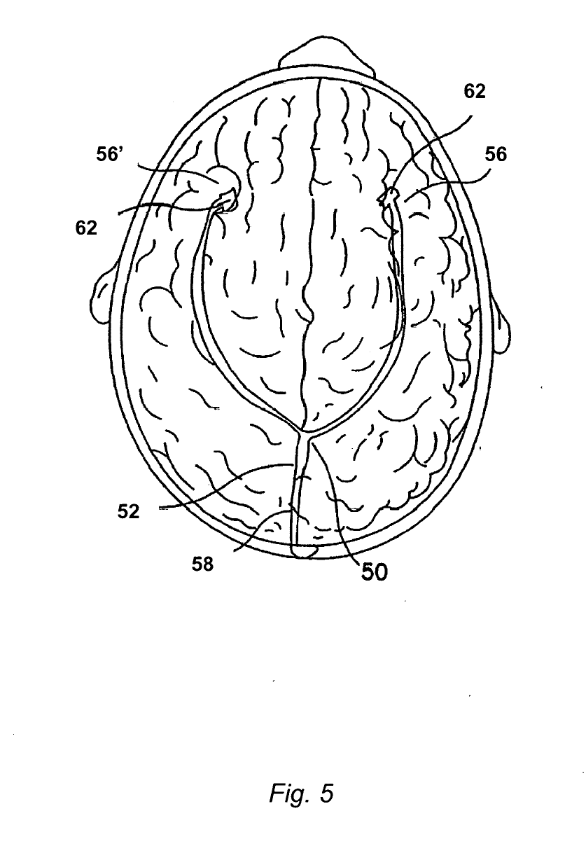

Bifurcated and branched catheter+pump systems are described in commonly-assigned U.S. Pat. No. 6,551,290. FIG. 4 illustrates a bifurcated catheter as implanted in an exemplary location of the human body, and for delivery of therapeutic protein formulation to each side of a patient's brain. FIG. 5 illustrates a top view of such a bifurcated catheter system as implanted and which provides for delivery of therapeutic protein formulation to each side of a patient's brain in accordance with the present invention. Referring to FIGS. 4 and 5, catheter 58 has a proximal end 54, and distal ends 62 and 62′. Distal ends 62 and 62′ are connected to catheter 58, which splits at a “Y” connector 50. In this particular embodiment, distal end 62 is positioned in the right anterior cerebral cortex 56, and distal end 62′ is positioned in the left anterior cerebral cortex 56′. Proximal end 54 is attached to device 20, which can be an implantable infusion pump. While two distal ends are shown, the present invention can have one or more than two distal ends. As further shown in the embodiment depicted in FIG. 5, catheter 58 has a catheter portion 52 downstream of device 20 and upstream of connector 50.

Neurological diseases for which any or all of the above-described embodiments for this system of treatment may find use include protein deficiency diseases, which include but are not limited to, inborn errors of metabolism selected from the group consisting of gangliosidosis (sphingolipidosis), glycoprotein disorders, glycogen storage diseases, mucolipidosis, mucopolysaccharidosis, cholesterol ester storage disease, farber lipogranulomatosis, galactosialidosis type I, galactosialidosis type II, neuronal ceroid lipofuscinosis (CLN1), other lysosomal storage diseases, and combinations thereof; and other protein deficiency diseases including Fragile X Syndrome and Parkinson's disease and combinations thereof.

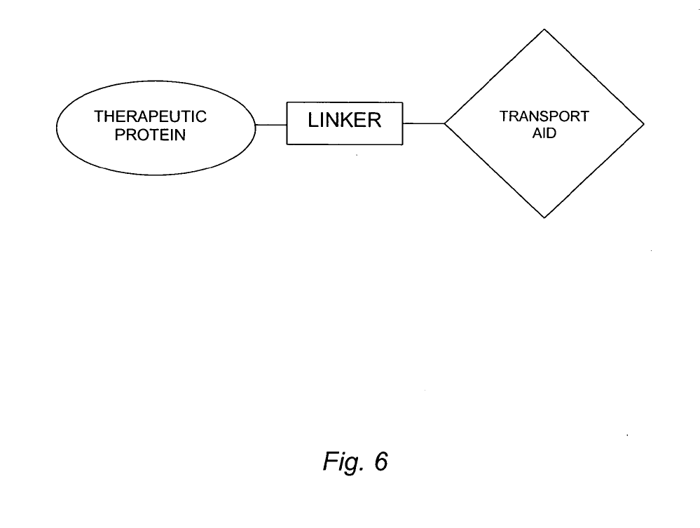

Generally, the therapeutic protein formulation, in accordance with the present invention, comprises proteins that have been formulated for enhanced cellular uptake. Such modified (i.e., enhanced) proteins generally comprise the therapeutic protein or proteins in which the patient is deficient or lacking (or for some reason inactive), and also a transport aid to which said therapeutic protein is bonded and which facilitates cellular uptake (e.g., endocytosis) of the therapeutic protein into CNS cells of the central nervous system. The transport aid can be any species that, when conjugated (i.e., associated) with a therapeutic protein of the present invention to form a therapeutic complex, enhances the ability of the therapeutic complex (relative to the therapeutic protein alone) to penetrate cell membranes. In some embodiments, the transport aid comprises at least a portion of a species selected from the group consisting of recombinant human melanotransferrin (p97), tetanus toxin fragment C (TTC), endogenous lectins, and combinations thereof. In some embodiments, the transport aid is biotin. The bonding of the therapeutic protein with the transport aid may or may not include a covalent bond, and said linker can be selected from the group consisting of peptide linkages, disulfide linkages, and combinations thereof. FIG. 6 illustrates a general manner in which therapeutic proteins can be linked with a transport aid using a linker species according to some embodiments of the present invention. In some or other embodiments, the linkage is a strepavidin-biotin complex, or engineered variant of an avidin or streptavidin and biotin binding pair, wherein the therapeutic protein is linked to either the avidin or the biotin species, and the transport aid is linked to the other of the avidin species or biotin species. For example, a therapeutic complex comprising a streptavidin and 2′-iminobiotin complex may be used to link the therapeutic protein with the transport aid in a pH-dependent manner, such that the therapeutic protein and transport aid remain operably linked at the neutral pH environment of the CSF, but become dissociated once taken up by cells into lysosomal compartments, or other acidic intracellular organelles. A streptavidin and 2′-iminobiotin complex with such pH-dependent affinity has been described by Athappilly and Hendrickson [Athappilly et al., “Crystallographic analysis of the pH-dependent finding of iminobiotin by streptavidin,” Protein Science, 6(6):1338-42 (1997)].

In some embodiments, the therapeutic protein formulation comprises one or more proteins. Such proteins, being deficient in patients being treated for neurological diseases/disorders of the central nervous system selected from the group consisting of protein deficiency diseases, enzyme deficiency diseases, lysosomal storage diseases, inborn errors of metabolism, and combinations thereof, include, but are not limited to, beta-glucosidase (glucocerebrosidase), acid sphingomyelinase, galactocerebrosidase, arylsulfatase A, saposin B, alpha-galactosidase A, beta-galactosidase, beta-hexosaminidase A, beta-hexosaminidase A and B, alpha-L-fucosidase, alpha-D-mannosidase, beta-D-mannosidase, N-aspartyl-beta-glucosaminidase, alpha-glucosidase, LAMP-2, glycogen branching enzyme, neuraminidase, phosphotransferase, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, beta-galactosidase, N-acetylgalactosamine 4-sulfatase, beta-glucuronidase, lysosomal acid lipase (acid cholesteryl ester hydrolase), acid ceramidase, N-acetyl-alpha-D-galactosaminidase, palmitoyl protein thioesterase, GDNF, Fragile-X mental retardation protein (FMRP), and combinations thereof.

In some embodiments of the present invention, the therapeutic protein formulation comprises one or more agents to maintain a physiologically acceptable pH when stored in a system reservoir (i.e., a pH or pH range that will not promote degradation of the therapeutic protein/enzyme) that may or may not be integrated with a system pump. In some or other embodiments, additional or other anti-degradation agents may be added to prevent dissociation of the proteins and/or protein complexes. This can be particularly relevant in embodiments where said reservoir is implantable and maintained at elevated (i.e., body) temperatures for long periods.

The delivery capacity and delivery rate of the therapeutic protein formulation via the catheter system is highly dependent on the particular therapy being administered and on patient needs. Furthermore, concentration of the therapeutic protein within the formulation must be considered when determining such delivery capacities or rates. Such variation in, and variability of, the concentration and delivery rate of the therapeutic protein formulation will be apparent to those of skill in the art.

### System for Providing Treatment of Neurological Diseases

Viewed differently, the present invention is directed to a system comprising: 1) a means of providing for a therapeutic protein formulation that facilitates (i.e., enhances) cellular uptake (e.g., endocytosis) of proteins within said formulation; and 2) a means of physically bypassing the blood-brain barrier so as to deliver the therapeutic protein formulation to target cells for the purpose of treating neurological diseases/disorders of the central nervous system.

Similarly, such neurological diseases include, but are not limited to, protein deficiency diseases, enzyme deficiency diseases, lysosomal storage diseases, inborn errors of metabolism, gangliosidosis (sphingolipidosis), glycoprotein disorders, glycogen storage diseases, mucolipidosis, mucopolysaccharidosis, cholesterol ester storage disease, farber lipogranulomatosis, galactosialidosis type I, galactosialidosis type II, neuronal ceroid lipofuscinosis (CLN1), Fragile X Syndrome, Parkinson's disease, and combinations thereof.

In such above-described embodiments, providing for a therapeutic protein formulation comprises: 1) identifying and selecting at least one appropriate protein material, appropriate for use in protein replacement therapy for a particular neurological disease/disorder of the central nervous system; and 2) conjugating, or otherwise associating, at least one transport aid to the said at least one appropriate protein material for facilitating enhanced cellular uptake (e.g., endocytosis).

Identifying and selecting the at least one appropriate protein material to provide for a therapeutic protein formulation, appropriate for use in protein replacement therapy for a particular neurological disease/disorder of the central nervous system, generally entails a suitable diagnosis accompanied by, possibly, one or more diagnostic tests. With inborn errors of metabolism or other genetic diseases, the positive diagnosis can be obtained by molecular analysis with the identification of a genetic mutation. When diagnosis confirms a particular protein deficient disease, such as one or more of those in TABLE 1(a-f), suitable protein(s) can be identified and selected. Lastly, the therapeutic protein is conjugated to a transport aid for the purpose of enhancing uptake of the protein/enzyme therapy by CNS cells.

As above, therapeutic proteins, for the purposes of providing for a therapeutic protein formulation, according to the present invention, include, but are not limited to, beta-glucosidase (glucocerebrosidase), acid sphingomyelinase, galactocerebrosidase, arylsulfatase A, saposin B, alpha-galactosidase A, beta-galactosidase, beta-hexosaminidase A, beta-hexosaminidase A and B, alpha-L-fucosidase, alpha-D-mannosidase, beta-D-mannosidase, N-aspartyl-beta-glucosaminidase, alpha-glucosidase, LAMP-2, glycogen branching enzyme, neuraminidase, phosphotransferase, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, beta-galactosidase, N-acetylgalactosamine 4-sulfatase, beta-glucuronidase, lysosomal acid lipase (acid cholesteryl ester hydrolase), acid ceramidase, N-acetyl-alpha-D-galactosaminidase, palmitoyl protein thioesterase, GDNF, FMRP, and combinations thereof.

To provide a solution or formulation comprising the at least one appropriate protein/enzyme, isolated quantities of the protein(s)/enzyme(s), generally in an aqueous medium, must be obtained using methods known in the art. Acceptable levels of solution pH for such formulations are those that generally maintain the integrity of the therapeutic protein/enzyme, i.e., resist degradation of the species within the formulation. Additionally, other anti-degradation agents may be added to prevent dissociation of the proteins and/or protein complexes.

To conjugate at least one transport aid to the said at least one appropriate therapeutic protein/enzyme material for the purpose of facilitating enhanced cellular uptake (e.g., endocytosis), a suitable transport aid(s) must be identified and selected possibly along with a suitable linker or linkers. Suitable transport aids are any species or species fragments which, when conjugated to the therapeutic enzyme/protein to form a therapeutic complex, enhances the ability of said resulting therapeutic complex to cross through the cell membrane and into cells of the CNS for the purpose of providing for protein replacement therapy, according to the present invention.

Conjugation, according to the present invention, is an association whereby the therapeutic protein/enzyme is linked, reversibly or otherwise, to a transport aid as described herein. Such a link generally requires a linker or linkage, wherein such linker or linkage may comprise covalent chemical bonding, and/or wherein it may comprise some other non-covalent association of a chemical, physical, and/or mechanical nature (e.g., hydrogen bonding).

For the purposes of providing for a therapeutic protein formulation, according to some embodiments of the present invention, said transport aid may comprise all or a portion of a species selected from the group consisting of recombinant human melanotransferrin (p97), tetanus toxin fragment C (TTC), endogenous lectins, biotin, and combinations thereof. Additionally, conjugating the at least one transport aid to the at least one appropriate protein/enzyme material may comprise a linker species, wherein said linker species may be selected from the group consisting of peptide linkages, disulfide linkages, and combinations thereof. Additionally or alternatively, the linker can be a streptavidin-biotin complex. In this latter case, the therapeutic protein (e.g., enzyme) is attached, covalently or otherwise, to either the streptavidin species or the biotin species, and the transport aid is attached to the other species of the complex with the attachment again being covalent or otherwise.

Streptavidin is a tetrameric protein that binds to biotin with an affinity that is among the highest displayed for noncovalent interactions between a ligand and a protein (Kₐ˜10¹³ M⁻¹). X-ray crystallographic studies of streptavidin have provided considerable insight into the structural origins of the high affinity of the streptavidin-biotin system. Streptavidin displays a number of commonly observed molecular recognition motifs in the interaction with biotin: these include hydrophobic and van der Waals dispersive interactions that are largely mediated by aromatic side chains of tryptophan (Trp) residues, hydrogen bonding networks mediated by donor-acceptor side chains, and disorder-to-order transitions mediated by the ordering of surface polypeptide loops upon ligand binding. See Chilkoti et al., “Site-directed mutagenesis studies of the high-affinity streptavidin-biotin complex: Contributions of tryptophan residues 79, 108, and 120,” Proc. Natl. Acad. Sci. USA, 92: 1754-1758 (1995).

Chemistries providing for conjugation, enhanced endocytosis, etc., are known in the art. Chian et al., “Insulin-like growth factor-1: tetanus toxin fragment C fusion protein for improved delivery of IGF-1 to the CNS,” Program No. 413.14, Abstract Viewer, Society for Neuroscience Annual Meeting (2003), have described a fusion protein of functional enzyme domain with tetanus toxin fragment C. Matthews et al., “A streptavidin-tetanus toxin C fragment fusion protein for the delivery of biotinylated molecules to neurons,” Program No. 733.18 Abstract Viewer, Society for Neuroscience Annual Meeting (2003), have described a carrier molecule comprising a tetanus toxin fragment C fusion with streptavidin, mixed with biotinylated enzyme. Dobrenis and Rattazzi, “Neuronal lysosomal enzyme replacement using fragment C of tetanus toxin,” Proc Natl Aced Sci USA, 89(6):2297-301 (1992), describe the coupling of functional enzyme with tetanus toxin fragment C using disulfide linkages. Beliveau et al., United States Patent Application Serial No. 20030129186, describe p97 protein conjugated to active agents. Allen et al., U.S. Pat. No. 5,433,946, describe endogenous lectins used as transport vehicles. See also: Larson et al., “Glial-derived neurotrophic factor:tetanus toxin fragment C fusion protein for targeted delivery of GDNF to neurons,” Program No. 299.5, Abstract Viewer, Society for Neuroscience Annual Meeting (2003); and Zirzow et al., “Delivery, distribution, and neuronal uptake of exogenous mannose-terminal glucocerebrosidase in the intact rat brain,” Neurochem Res., 24(2):301-305 (1999).

Pump, catheter, reservoir, and programming devices can be as described above, or different such that they provide for the delivery of therapeutic protein formulation, for the treatment of protein deficiency diseases, wherein such delivery can be programmably rate-controlled for the purpose of providing a controlled dosing regimen and allowing for chronic delivery for long-term therapies.

### Methods for Treating Neurological Diseases

In general terms, the present invention is directed to a method of physically delivering one or more therapeutic protein formulations across the blood-brain barrier (BBB) to the central nervous system (CNS), via one or more implantable catheters, for the purpose of treating neurological diseases of the central nervous system. The therapeutic proteins of the present invention can be delivered to intrathecal regions, intracerebroventricular regions, intraparenchymal regions, or to various combinations of these.

Such neurological diseases for which these methods offer treatment include, but are not limited to, protein deficiency diseases, enzyme deficiency diseases, lysosomal storage diseases, inborn errors of metabolism, gangliosidosis (sphingolipidosis), glycoprotein disorders, glycogen storage diseases, mucolipidosis, mucopolysaccharidosis, cholesterol ester storage disease, farber lipogranulomatosis, galactosialidosis type I, galactosialidosis type II, neuronal ceroid lipofuscinosis (CLN1), Fragile X Syndrome, Parkinson's disease, and combinations thereof.

In some embodiments of the present invention, an injection port is provided and connected to the implantable catheter for the purpose of administering a therapeutic protein formulation to the central nervous system. In some or other embodiments, a reservoir is used to store a quantity of the therapeutic protein formulation and a pump can be used to direct the therapeutic protein formulation from the reservoir or other source, through the one or more catheters, and into one or more target regions. In some embodiments, an integrated implantable pump+reservoir is refillable through a subcutaneous inlet.

In some embodiments of the present invention, intracerebroventricular catheters, such as the Medtronic Model 8506 Intracerebroventricular Access Port and Model 8770 Intracerebroventricular Catheter (Medtronic Inc., Minneapolis, Minn.), are used to direct the delivery of a bolus of the enzymes or proteins that have been formulated for rapid uptake into the CNS cells. In some or other embodiments, an intraparenchymal catheter, such as Medtronic Model 10541, is used for intraparenchymal delivery.

In some embodiments of the present invention, an implantable catheter comprising an access port is used. Such access ports can be of a wide variety of suitable inlet or injection ports including, for example, the one depicted in FIG. 9.

In some embodiments of the present invention, a Medtronic SynchroMed Infusion System, or similar pump system, is used for intrathecal, intracerebroventricular, and/or intraparenchymal delivery of therapeutic protein formulation. Such systems comprise an implantable, programmable pump; a catheter; and an external programmer. Suitable catheters include, but are not limited to, Medtronic InDura 1P Intrathecal Catheter Model 8709, and the InDura Free-flow Intrathecal Catheter Model 8711. Suitable pumps include, but are not limited to, the implantable MiniMed or SynchroMed series of pumps by Medtronic Inc. Suitable models include, but are not limited to, 8626-18, 8626L-18, 8627-18, 8627L-18, 8626-10, 8626L-10, wherein all of these pumps have an integral reservoir and wherein the pump is refilled by using a needle and syringe to inject the drug through the skin into the drug reservoir. Programming such catheter systems to deliver a specific therapeutic protein formulation at a certain rate or programmed rate ramp can be done with the Medtronic Programmer. Such programmable rate provides for a controlled dosing regimen, allowing for the avoidance of toxic side-effects of treatment.

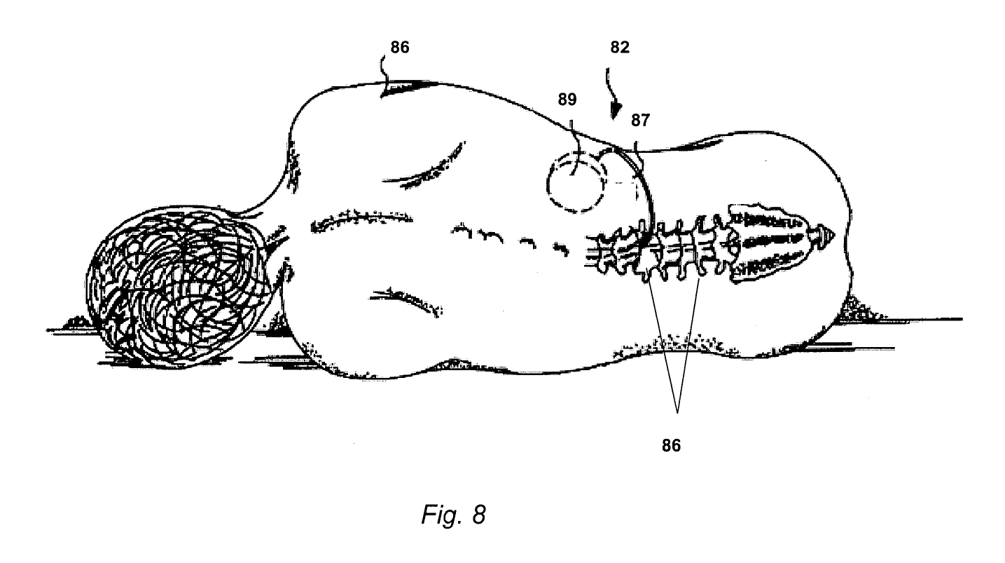

In some embodiments of the present invention, the catheter system is implanted in a patient for intrathecal delivery of therapeutic protein formulation. FIG. 8 shows the general placement of catheter system 22 in relation to the body 26, illustrating one possible catheter placement, according to at least one embodiment of the present invention. In FIG. 8, an Implantable Infusion Pump (IIP) 89 is surgically implanted subcutaneously in the abdominal region of the body 86. Catheter 87 is tunneled subcutaneously and the distal end and tip (obscured from view and not shown) and is positioned between vertebrae 86 to infuse the therapeutic protein formulation into the intrathecal space.

Suitable catheter systems comprising pumps are described in commonly-assigned U.S. Pat. Nos. 6,093,180 and 6,594,880. The use of such systems for the general treatment of neurodegenerative disorders is described in commonly-assigned U.S. Pat. No. 5,814,014, and for the treatment of Alzheimer's disease in commonly-assigned U.S. Pat. Nos. 5,846,220; 6,056,725; and 6,503,242.

Alternatively, a non-integrated reservoir may be used. Alternate devices for delivery of therapeutic protein formulation to intrathecal, intracerebroventricular, and/or intraparenchymal regions of the central nervous system include, but are not limited to Ommaya reservoirs like those described in U.S. Pat. Nos. 5,222,982 and 5,385,582, and United States Patent Application Serial No. 20020142985.

In some embodiments, the catheter is a bifurcated or multiply branched catheter like that described in U.S. Pat. No. 6,551,290. Such a bifurcated (or branched) catheter allows for the delivery of protein formulation to two separate target regions using a single catheter. In some embodiments, multiple bifurcated catheters and/or multiply branched catheters are used in combination with one or more pump+reservoir systems.

A protein formulation must be generated that is capable of treating protein deficient diseases according to the present invention. Such a protein formulation generally comprises a therapeutic protein that treats the protein deficiency—generally alleviating a genetically-induced disease/disorder. In some embodiments, the therapeutic proteins are enzymes. Such therapeutic proteins include, but are not limited to, beta-glucosidase (glucocerebrosidase), acid sphingomyelinase, galactocerebrosidase, arylsulfatase A, saposin B, alpha-galactosidase A, beta-galactosidase, beta-hexosaminidase A, beta-hexosaminidase A and B, alpha-L-fucosidase, alpha-D-mannosidase, beta-D-mannosidase, N-aspartyl-beta-glucosaminidase, alpha-glucosidase, LAMP-2, glycogen branching enzyme, neuraminidase, phosphotransferase, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, beta-galactosidase, N-acetylgalactosamine 4-sulfatase, beta-glucuronidase, lysosomal acid lipase (acid cholesteryl ester hydrolase), acid ceramidase, N-acetyl-alpha-D-galactosaminidase, palmitoyl protein thioesterase, GDNF, FMRP, and combinations thereof.

The therapeutic proteins of the present invention can be delivered to intrathecal regions, intracerebroventricular regions, intraparenchymal regions, or a combination of these, using a single catheter, a branched and/or bifurcated catheter, or one or multiple combinations of single and/or bifurcated and/or branched catheters—all relying on one or more pumps and reservoirs containing one or more therapeutic protein formulations for treating one or more protein deficiency diseases.

The methods and systems of the present invention generally rely on one or more catheters to physically deliver therapeutic proteins across the blood-brain barrier (BBB) to the central nervous system for uptake by CNS cells. Such protein delivery provides for treatment for a number of enzyme-deficient diseases. However, acknowledging that the blood-brain barrier is not the only obstacle to transcytosis of these proteins into brain cells, as such transcytosis does not take place readily, the proteins must generally be “coaxed” into the cells by chemically modifying them with a transport aid.

Generally, the methods and systems of the present invention comprise a reservoir to store a quantity of a therapeutic protein formulation, as well as a pump to force the protein formulation through the catheter to a targeted delivery area. In some embodiments, one or both of the reservoir and pump are implantable, i.e., surgically deposited inside the body of a patient. In some embodiments, one or both of the reservoir and pump are partially implantable (i.e., partially implanted). In some embodiments, the pump is programmable so as to be capable of altering the protein delivery rate in some predefined manner. This can be important, especially in the initial stages of treatment, so as to allow for the various other metabolic processes within the body to achieve an equilibrium with the newly administered therapy. In some embodiments, the reservoir is integrated with the pump.

To enter most CNS cells, the therapeutic proteins within said therapeutic protein formulation must generally be modified so as to facilitate uptake by CNS cells. To do this, the therapeutic proteins must be modified (e.g., enhanced). Such modified proteins generally comprise a therapeutic protein or enzyme, as described above, conjugated or associated with a transport aid. Such a transport aid is generally a species or species fragment that undergoes cellular uptake (e.g., transcytosis into the cells) relatively easily. Such transport aids can be conjugated either covalently or noncovalently via a linker species, as shown in FIG. 6. In some embodiments, the transport aid is a protein sequence integrated into the protein. In some embodiments, the modified protein is a fusion protein.

In some embodiments, said transport aid comprises at least a portion of a species selected from the group consisting of recombinant human melanotransferrin (p97), tetanus toxin fragment C (TTC), biotin, endogenous lectins, and combinations thereof. The linker species, according to some embodiments of the present invention, is selected from the group consisting of peptide linkages, disulfide linkages, and combinations thereof. Additionally or alternatively, the linker can be a streptavidin-biotin complex. In this latter case, the therapeutic protein (e.g., an enzyme) is attached, covalently or otherwise, to either the streptavidin species or the biotin species, and the transport aid is attached to the other species of the complex with the attachment again being covalent or otherwise.

Alternatively, the present invention is also directed to methods of using the systems described herein for treating neurological diseases of the central nervous system. Such neurological diseases include, but are not limited to, protein deficiency diseases, enzyme deficiency diseases, lysosomal storage diseases, inborn errors of metabolism, gangliosidosis (sphingolipidosis), glycoprotein disorders, glycogen storage diseases, mucolipidosis, mucopolysaccharidosis, cholesterol ester storage disease, farber lipogranulomatosis, galactosialidosis type I, galactosialidosis type II, neuronal ceroid lipofuscinosis (CLN1), Fragile X Syndrome, Parkinson's disease, and combinations thereof.

The present invention is novel in the use of an intrathecal, intracerebroventricular, and/or intraparenchymal catheter system for delivery of a protein, modified for enhanced cellular uptake, directly to the central nervous system, and the use of molecular modifications and/or carriers (transport aids) that provide this enhanced cellular uptake of the protein.

Presently, the use of implantable catheters for long-term delivery of modified (i.e., enhanced) proteins to the central nervous system via intrathecal, intracerebroventricular, or intraparenchymal delivery has not been addressed. An essential aspect of the present invention is not only the use of proteins modified or formulated for optimal cellular uptake, but also the use implantable delivery systems for chronic intrathecal, intracerebroventricular, and/or intraparenchymal delivery of the protein formulation. An additional aspect of the present invention is the use of dosing regimens and features of programmable pumps to ensure gradual introduction of the missing enzyme at a rate slow enough to avoid toxic side effects.

The present invention addresses and helps to overcome a number of problems in the therapeutic treatment of genetically-based protein deficiency diseases. Such problems include, but are not limited to, delivery of a missing or deficient protein to the central nervous system, delivery of a missing or deficient (in concentration or activity) protein for chronic treatment, effective dose delivery of a missing or deficient protein, safe delivery of a missing or deficient protein, control of delivery of a missing or deficient protein.

To deliver a missing or deficient protein into the central nervous system tissues of a patient suffering from an inborn error of metabolism or other defects which cause a protein deficiency, and thereby treat the neurological consequences of the enzyme or protein deficiency, the present invention concerns a device for delivery of proteins into the central nervous system for treatment of neuropathic diseases caused by the lack of the protein, using hardware similar to that described by Elsberry and Rise in U.S. Pat. No. 5,814,014. As mentioned previously herein, according to some embodiments of the present invention, a possible device for delivery of a quantity of the missing protein/enzyme into the central nervous system that is mentioned in the prior art (e.g., in United States Patent Application Serial No. 20020142985) is the “Ommaya reservoir,” which is described in U.S. Pat. Nos. 5,222,982 and 5,385,582. The latter patents for the Ommaya reservoir itself do not claim treatment of inborn errors of metabolism as a use for the device. The present invention differs, however, in that it also provides for a means of regulating the dosage of the enzyme, such as by use of the programmable features of an implantable drug pump. Such dosing regulation is generally an important aspect of such therapy, particularly in the early stages of treatment.

In order to chronically deliver a missing protein/enzyme, for therapeutic purposes, to a patient over the course of his/her lifetime, some embodiments of the present invention utilize a refillable implantable pump. Future therapies may further comprise the use of a gene therapy approach, used alone or in concert with the methods and systems of the present invention.

In order to deliver an effective dose of therapeutic protein/enzyme, some embodiments of the present invention can provide for the protein to be formulated or co-administered with other molecules in a manner that will optimize cellular uptake of the delivered enzyme by cells of the central nervous system. While others have disclosed methods for formulating enzymes for this purpose, specified physical delivery methods or devices for such formulations, such as the implantable pumps and catheters of the systems and methods described herein, have heretofore not been addressed.

In order to deliver a safe dose of the therapeutic protein/enzyme, some embodiments of the present invention provide for the regulation of the dosage of the delivered enzyme, particularly in the early stages of treatment, using dosing regimens and programmable features of an implantable pump, to ensure that the patient does not experience neurological damage as an unintended consequence of a bolus delivery of the missing or deficient protein/enzyme.

In order to ensure the delivery of an appropriate amount of the therapeutic protein/enzyme, according to some embodiments of the present invention, variations of the catheter+pump system described herein can permit the programmed release of a therapeutic protein formulation into the central nervous system. The programmed level can be determined by cerebrospinal fluid enzyme level assessment or by the known historical level based on the patient's specific genetic mutation and the patient's physical characteristics (e.g., height, weight, genetic sequence of the patient's gene encoding for the protein to be delivered, etc.). This allows the time between pump filling to be maximized while maintaining safe and effective levels of delivery.

As described herein, in some embodiments the present invention is also directed to methods of treating inborn errors of metabolism. Some such methods comprise the steps of: a) formulation of at least one species of therapeutic protein/enzyme with molecular domains or molecular carriers (transport or transcytosis aids) to enhance the uptake of the enzyme into CNS cells; and b) the chronic delivery of the formulated therapeutic enzyme or protein using a dosing schedule designed to provide a therapeutic benefit to the patient without incurring toxic effects, and wherein delivery of the therapeutic enzyme or protein is accomplished via an implanted catheter system positioned so as to deliver the composition to the central nervous system tissue or cerebral spinal fluid of the patient.

The present invention incorporates a number of advantages over presently known devices, systems or processes. These advantages include:
Extension of the benefits of protein replacement therapies for inborn errors of metabolism to the neurological manifestations of the disease. Protein replacement therapy, as currently practiced, does not treat these manifestations and there are no known cures.
Improvement in the efficacy of protein delivery to the central nervous system by virtue of using protein modifications that enhance uptake of the protein into the cells of the central nervous system.
Improvement in the safety of protein delivery to the central nervous system by virtue of controlling the dosage and rate of administration to minimize potential toxic side-effects that may arise using bolus delivery of enzymes.
Avoidance of the potential disadvantages of gene therapy (potential immune reaction to viral vectors, potential loss of gene expression, limited “coverage” of the central nervous system by the vector, inability to regulate the dosage of the protein resulting from the gene therapy), and undesired genetic mutations.

A primary use for the present invention is for the medical treatment of the neurological consequences of an inborn enzyme deficiency. The technique of modifying the therapeutic proteins to be delivered intrathecally, intracerebroventricularly, or intraparenchymally for purposes of improving uptake by neurons may also have application for delivery of other types of proteins for other types of diseases. For example, Larsen et al. [Larsen et al., “Glial-derived neurotrophic factor:tetanus toxin fragment C fusion protein for targeted delivery of GDNF to neurons,” Program No. 299.5, Abstract Viewer, Society for Neuroscience Annual Meeting (2003)] have reported on the development of a tetanus toxin fragment C fusion protein with glial-derived neurotrophic factor (TTC:GDNF) to improve upon the uptake of GDNF by neurons. Thus, a related use of the technology of this invention would be to deliver a TTC:GDNF protein to the central nervous system using an implantable pump and an intracerebroventricular, intrathecal, or intraparenchymal catheter system. The result may be an improved treatment of Parkinson's disease, beyond that attainable with an infusion of unmodified GDNF. Another possible application of this invention is for the treatment of Fragile X Syndrome by the delivery of the protein which is lacking and causes this disease.

The following example is included to demonstrate particular embodiments of the present invention. It should be appreciated by those of skill in the art that the systems and methods disclosed in the example which follows merely represent exemplary embodiments of the present invention. However, those of skill in the art should, in light of the present disclosure, appreciate that many changes can be made in the specific embodiments described and still obtain a like or similar result without departing from the spirit and scope of the present invention.

### Example

This Example serves to illustrate certain exemplary embodiments of the present invention that comprise: systems providing for the chronic delivery of a therapeutic protein formulation to intraparenchymal, intracerebroventricular, and intrathecal regions of the central nervous system; and methods of using such systems for the treatment of neurological diseases/disorders of the central nervous system.

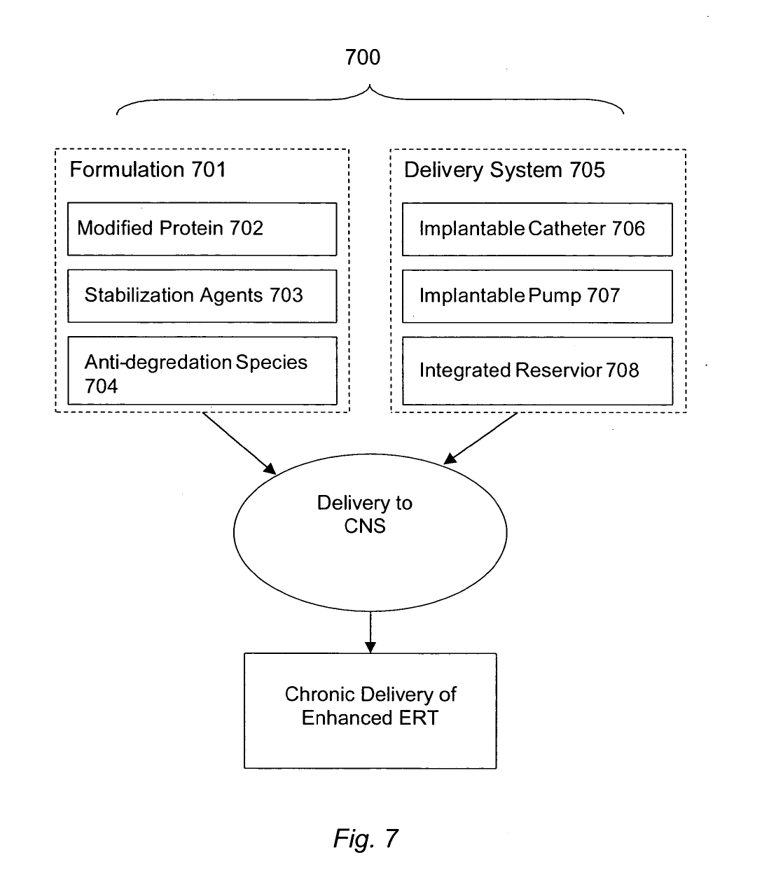

In this particular Example, the system provides treatment for Fragile X Syndrome by way of enhanced enzyme replacement therapy. Referring to FIG. 7, such a system 700 comprises: a therapeutic protein formulation 701 that in turn comprises a quantity of modified protein 702; one or more stabilization agents 703; and, optionally, one or more anti-degradation species 704; and wherein the therapeutic protein formulation provides for enhanced protein replacement therapy. System 700 further comprises a delivery system (subsystem) 705 comprising an implantable catheter 706; and implantable, programmable pump 707; and a refillable reservoir 708 integrated with the pump 707.

Use of System 700 for delivering therapeutic protein formulation (comprising protein FRMP, modified for optimal cellular uptake by CNS cells) as protein replacement therapy entails the intraparenchymal, intracerebroventricular, and/or intrathecal placement of a catheter 706, wherein the catheter is bifurcated, allowing for delivery to multiple CNS regions with a single catheter. The therapeutic protein formulation is pumped from integrated implantable reservoir 708, through catheter 706, and into the central nervous system (intrathecal, intracerebroventricular, and/or intraparenchymal regions) by way of implantable pump 707. Delivery is programmable such that in the early stages of treatment the dosing is lower, and then slowly ramped up to a constant maintenance delivery dosage. Such programmable ramping provides the body's metabolic system time to equilibrate to the therapy.

All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

1-76. (canceled)

77. A method comprising the step of:
physically delivering a therapeutic protein formulation selected to treat a neurological disease of the central nervous system via an implantable catheter system across the blood brain barrier of a patient with the neurological disease, wherein the therapeutic protein formulation comprises proteins that have been modified for enhanced cellular uptake.

78. The method of claim 77, wherein the therapeutic protein formulation is delivered in a manner selected from the group consisting of intrathecally, intraparenchymally, intracerebroventricularly, and combinations thereof.

79. The method of claim 77, wherein the neurological diseases to be treated are selected from the group consisting of lysosomal storage diseases, protein deficiency diseases, enzyme deficiency diseases, inborn errors of metabolism, neurodegenerative diseases, and combinations thereof.

80. The method of claim 77, wherein said neurological diseases are inborn errors of metabolism selected from the group consisting of gangliosidosis, sphingolipidosis, glycoprotein disorders, glycogen storage diseases, mucolipidosis, mucopolysaccharidosis, cholesterol ester storage disease, farber lipogranulomatosis, galactosialidosis type I, galactosialidosis type II, neuronal ceroid lipofuscinosis, and combinations thereof.

81. The method of claim 77, wherein said neurological diseases are selected from the group consisting of Fragile X Syndrome, Parkinson's disease, Alzheimer's disease, and combinations thereof.

82. The method of claim 77, wherein the therapeutic protein formulation comprises enzymes providing for enzyme replacement therapy.

83. The method of claim 82, wherein the enzymes are selected from the group consisting of beta-glucosidase, glucocerebrosidase, acid sphingomyelinase, galactocerebrosidase, arylsulfatase A, saposin B, alpha-galactosidase A, beta-galactosidase, beta-hexosaminidase A, beta-hexosaminidase A and B, alpha-L-fucosidase, alpha-D-mannosidase, beta-D-mannosidase, N-aspartyl-beta-glucosaminidase, alpha-glucosidase, LAMP-2, glycogen branching enzyme, neuraminidase, phosphotransferase, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, beta-galactosidase, N-acetylgalactosamine 4-sulfatase, beta-glucuronidase, lysosomal acid lipase, acid cholesteryl ester hydrolase, acid ceramidase, N-acetyl-alpha-D-galactosaminidase, palmitoyl protein thioesterase, and combinations thereof.

84. The method of claim 77, wherein the therapeutic protein formulation comprises proteins selected from the group consisting of GDNF, FMRP, and combinations thereof.

85. The method of claim 77, wherein at least some of the proteins within said therapeutic protein formulation are modified so as to comprise a transport aid that provides for enhanced cellular uptake of said modified proteins.

86. The method of claim 85, wherein said modified proteins are modified by incorporating into their structure amino acid sequences providing for an intrinsic transport aid.

87. The method of claim 86, wherein said modified proteins are fusion proteins.

88. The method of claim 85, wherein said modified proteins are modified by conjugating to them a transport aid that facilitates the cellular uptake of said therapeutic protein.

89. The method of claim 88, wherein the transport aid comprises at least a portion of a species selected from the group consisting of recombinant human melanotransferrin, p97, tetanus toxin fragment C, endogenous lectins, biotin, and combinations thereof.

90. The method of claim 88, wherein the modification by conjugating comprises at least one linker species existing between said therapeutic protein and said transport aid.

91. The method of claim 90, wherein said linker is selected from the group consisting of peptide linkages, disulfide linkages, and combinations thereof.

92. The method of claim 88, wherein the conjugation is non-covalent.

93. The method of claim 90, wherein said linker is a streptavidin-biotin complex.

94. The method of claim 77, wherein said therapeutic protein formulation is formulated to help maintain the integrity and activity of the protein formulation.

95. The method of claim 77, wherein said therapeutic protein formulation is introduced into the said implantable catheter system via an injection port.

96. The method of claim 77, wherein said therapeutic protein formulation is held in a reservoir.

97. The method of claim 96, wherein the reservoir is implantable and refillable.

98. The method of claim 77, the implantable catheter system further comprising a pump to direct therapeutic protein formulation through said implantable catheter and into a target region.

99. The method of claim 98, wherein the pump comprises an integrated reservoir.

100. The method of claim 98, wherein the pump is implantable.

101. The method of claim 77, wherein said implantable catheter system comprises at least one bifurcated catheter.

102. The method of claim 98, wherein the pump provides for a programmable delivery rate of the therapeutic protein formulation, and wherein the delivery rate is selected based on factors selected from the group consisting of specific neurological disease, genetic sequence of the patient's gene encoding for the protein to be delivered, body weight, and combinations thereof.

103-128. (canceled)
